(19) United States
(12) Patent Application Publication
(10) Pub. No.: US 2023/0053208 A1
(43) Pub. Date: Feb. 16, 2023

(54) Nasal Right Angle Endotracheal (RAE) Tube Protective Tip and Method of Use

(71) Applicant: Nosrat Khajavi, Las Vegas, NV (US)

(72) Inventor: Nosrat Khajavi, Las Vegas, NV (US)

(21) Appl. No.: 17/399,108

(22) Filed: Aug. 11, 2021

(51) Int. Cl. A61M 16/04; A61M 16/20

(52) U.S. Cl. CPC A61M 16/0461; A61M 16/0402; A61M 16/20; A61M 16/045; A61M 2205/02

(57) ABSTRACT

A tube protective sheath or tip is provided for use with a nasal RAE tube in order to minimize bleeding and trauma often associated with passage of a nasal RAE tube through the nasal cavity. The tube protective sheath or tip includes a hollow cylindrical portion that extends and fits snugly over the tube tip of the nasal RAE tube. A tapered, pointed, or rounded distal portion is connected to the hollow cylindrical portion. As the nasal RAE tube is inserted through the patient's nasal cavity, the nasal RAE tube protective sheath or tip spreads the passages gently and smoothly, allowing the nasal RAE tube to follow it into the pharynx. Strings or extensions are attached to the tube protective sheath or tip and are used to prevent loss of the protective tip in the patient's nasal cavity, pharynx, or trachea during the intubation process.

### BACKGROUND

### Field of Invention

Embodiments of the present invention described herein generally relate to a Nasal Right Angle Endotracheal (RAE) Tube Protective Tip and a Method of Using the same. The Nasal RAE Tube Protective Tip is designed to minimize or eliminate the bleeding that often results from the intubation of patients using an ordinary Nasal RAE Tube. The Nasal RAE Tube Protective Tip is configured to prevent loss of the protective tip in the patient's nasal cavity, pharynx, or trachea during the intubation process, while allowing medical personnel to establish and maintain the patient's airway.

### Related Art

During surgeries performed in or around the oral cavity or mouth, such as orthodontic surgery, reconstructive surgery of the face, facial trauma surgery, surgery involving the head and neck, mouth, and jaws (maxillofacial surgery), as well as during facial cosmetic surgery, medical personnel must secure the patient's airway and protect the patient's lungs. A nasal RAE tube, then, is a tube that is inserted into the trachea for the primary purpose of establishing and maintaining a patient's airway, as well as preventing any blood or other fluids from getting into the lungs, and to ensure the adequate exchange of oxygen and carbon dioxide. This nasal RAE tube is always inserted through the nose, through the nasal passages, through the pharynx and larynx, and into the trachea beyond the vocal cords. Magill's forceps are often used to guide the tip of the endotracheal tube through the vocal cords and into the trachea. Once the tip of the endotracheal tube is positioned within the trachea, an inflatable cuff attached to the nasal RAE tube is inflated to provide a seal between the endotracheal tube and the trachea, so that an adequate flow of air to the lungs is ensured, and to seal the lungs from the oral cavity, thereby preventing any fluids from getting into the lungs.

Many times, despite the best efforts of medical personnel, and for a variety of reasons such as a deviated septum, small nasal cavity, obstructions, hyperplasia of the turbinates, and etcetera, the tips of known nasal RAE tubes traumatize the nasal passages, pharynx, larynx, and/or vocal cords, causing bleeding. This is caused by the tip of known nasal RAE tubes, which may be excessively rigid, wide, blunt, and/or flared, characteristics that are necessitated by the function of the nasal RAE tube. This can not only make it challenging to pass the endotracheal tube into the trachea because of blood obscuring the airway and the endotracheal tube, but the bleeding can also be severe enough to cause harm to the patient.

Accordingly, there is an unmet need for a nasal RAE tube that avoids traumatizing the nasal cavity, pharynx, larynx, and/or vocal cord while being inserted into the trachea for the purpose of establishing and maintaining a patient's airway, and ensuring the adequate exchange of oxygen and carbon dioxide.

### SUMMARY

Embodiments described herein relate to a Nasal Right Angle Endotracheal (RAE) Tube Protective Tip and Method of Use thereof. The Nasal RAE Tube Protective Tip is designed to minimize or eliminate bleeding and trauma of patients' nasal passages and pharynx during the intubation process. Further, the Nasal RAE Tube Protective Tip is configured to prevent loss of the protective tip in the patient's nasal cavity, pharynx, or trachea during the intubation process, while allowing the medical personnel to establish and maintain the patient's airway.

The present invention includes a protective sheath or tip that is provided with a hollow cylindrical portion and a tapered, pointed, or rounded distal portion. In at least one non-limiting exemplary embodiment of the Nasal RAE Tube Protective Tip, the hollow cylindrical portion is about 1 to 2 centimeters long, and the tapered, pointed, or rounded distal portion is about 1 to 3 centimeters long. The protective sheath or tip may be made from silicone, rubber, or plastic. It may be designed in some embodiments to be disposable or reusable in other embodiments. The hollow cylindrical portion extends over the tip of the nasal RAE tube and fits snugly thereto. In some embodiments of the present invention, the hollow cylindrical portion extends only over the tip of the nasal RAE tube, whereas in other embodiments of the present invention, the hollow cylindrical portion may extend partially or fully over the cuff of the nasal RAE tube.

The tapered, pointed, or rounded distal portion may be solid or hollow, or a combination of both, as needed to provide the level of stiffness and resiliency required. That is to say, the tapered, pointed, or rounded distal portion is semi-rigid, yet pliable, so that it is sufficiently rigid to avoid bending over when pushed into the nasal cavity, yet soft enough to prevent tissue damage or trauma. If the tapered, pointed, or rounded distal portion is hollow or partially hollow, the hollow space within the distal portion may or may not be in fluid communication with the interior of the hollow cylindrical portion of the nasal RAE tube protective sheath or tip. In this way, the tube of the nasal RAE tube may be used to pressurize or depressurize the hollow space within the distal portion as needed to help manipulate the nasal RAE tube with the Nasal RAE Tube Protective Tip through the patient's nasal cavity.

As the nasal RAE tube is inserted through the patient's nasal cavity, the nasal RAE tube protective sheath or tip spreads the passages gently and smoothly, allowing the nasal RAE tube to follow it into the pharynx, and minimizing trauma and bleeding of the nasal passages. In some embodiments of the Method of Use of the Nasal RAE Tube Protective Tip of the present invention, the protective tip may be lubricated prior to engaging it to the nasal RAE tube. In such an embodiment, the nasal RAE tube protective tip may be provided pre-lubricated in a package, so that medical personnel may simply open the packaging and engage the pre-lubricated nasal RAE tube protective tip with the nasal RAE tube.

The Nasal RAE Tube Protective Tip of the present invention may be provided in several different sizes or diameters to engage with the tube tips of different size or diameter nasal RAE tubes. In this case, the Nasal RAE Tube Protective Tip of the present invention may be provided in different colors that are coded to indicate the size or diameter of nasal RAE tubes with which the Nasal RAE Tube Protective Tip is configured to engage. Additionally, each of the colors chosen for color coding of the Nasal RAE Tube Protective Tip may be chosen to provide sufficient contrast with blood to ensure that medical personnel are able to locate and remove the Nasal RAE Tube Protective Tip even in the event that there is blood in the oral cavity.

Strings or extensions may be attached to the nasal RAE tube protective sheath or tip, and may be provided with loops at their distal ends. The strings or extensions and loops are used to ensure that the nasal RAE tube protective sheath or tip is removed from the patient's nasal passages if the nasal RAE tube is withdrawn, and/or to ensure that the nasal RAE tube protective sheath or tip does not inadvertently obstruct the trachea during the removal process. In at least one non-limiting exemplary embodiment of the Nasal RAE Tube Protective Tip, the strings or extensions are about 15 to 25 centimeters in length and are positioned symmetrically about the centerline of the nasal RAE tube protective sheath or tip.

In another non-limiting exemplary embodiment of the Nasal RAE Tube Protective Tip, the strings or extensions may be positioned asymmetrically and used in combination with one or more perforations or similar separation features. The one or more perforations or separation features may be positioned longitudinally along the hollow cylindrical portion or otherwise. In this way, when the strings or extensions and loops are pulled, the hollow cylindrical portion of the nasal RAE tube protective sheath or tip splits, and the nasal RAE tube protective sheath or tip may then be withdrawn using the strings or extensions. The nasal RAE tube protective sheath or tip remains intact with its hollow cylindrical portion and its tapered, pointed, or rounded distal portion all connected to the strings or extensions, thereby ensuring that the nasal RAE tube protective sheath or tip is fully removed. It is further contemplated that at least one of the strings or extensions may also function as an inflation tube that is connected to a hollow embodiment of the tapered, pointed, or rounded distal portion, so that the tapered, pointed, or rounded distal portion may be inflated or deflated as needed.

In use, the nasal RAE tube with the nasal RAE tube protective sheath or tip in place is passed through the patient's nasal cavity into the patient's pharynx at the back of the patient's oral cavity. If there is sufficient resistance to insertion of the nasal RAE tube, for non-limiting example because of a deviated septum, small nasal cavity, obstructions, and etcetera, such that in the judgment of the medical personnel the nasal RAE tube cannot be safely inserted by way of the patient's nasal cavity, having tried both sides, the nasal RAE tube may be withdrawn. In this situation, the strings or extensions are used to ensure that the nasal RAE tube protective sheath or tip is removed along with the nasal RAE tube.

Once the tube tip appears at the back of the patient's oral cavity, the medical personnel inserts Magill's forceps and removes the nasal RAE tube protective sheath or tip from the tube tip. The nasal RAE tube protective sheath or tip may then be withdrawn by way of the patient's oral cavity, in which case the strings or extensions and loops may follow through the patient's nasal cavity. Alternately, the nasal RAE tube protective sheath or tip may then be withdrawn back through the patient's nasal cavity. Still alternately, once the nasal RAE tube protective sheath or tip is secured using the Magill's forceps, the medical personnel may decide to sever the strings or extensions so that the severed strings or extensions are removed back through the patient's nasal cavity, and the nasal RAE tube protective sheath or tip is removed through the patient's oral cavity. As Magill's forceps are currently a standard and necessary device for nasal intubations, no additional instruments are required to accomplish this procedure.

Still alternately, the nasal RAE tube protective sheath or tip may be left in place on the tube tip of the nasal RAE tube until the tube tip passes the vocal cords and enters the trachea. Prior to inflation of the cuff, the nasal RAE tube protective sheath or tip is then withdrawn using the strings or extensions and loops. In order to facilitate this, the medical personnel may use an embodiment of the nasal RAE tube protective sheath or tip having the one or more perforations or similar separation features, so that when the strings or extensions and loops are pulled, the hollow cylindrical portion of the nasal RAE tube protective sheath or tip splits, and the nasal RAE tube protective sheath or tip is withdrawn.

According to one embodiment of the Nasal RAE Tube Protective Tip and Method of Use thereof, a tube protective sheath or tip for use with a Nasal RAE Tube includes a hollow cylindrical portion configured to extend and fit snugly over the tube tip of a nasal RAE tube. A tapered, pointed, or rounded distal portion is connected to the hollow cylindrical portion. String or extension are attached to the hollow cylindrical portion or the tapered, pointed, or rounded distal portion.

According to another embodiment of the Nasal RAE Tube Protective Tip and Method of Use thereof, a nasal RAE tube has a tube protective sheath or tip. The nasal RAE tube includes a tube, an air supply connection connected to the tube, and a tube tip for insertion into the trachea of a patient by way of the patient's nasal cavity. The nasal RAE tube further includes a cuff for inflatably sealing against the patient's trachea. A cuff inflator valve is connected to the cuff by way of a cuff inflator tube. The tube protective sheath or tip includes a hollow cylindrical portion configured to extend and fit snugly over the tube tip of a nasal RAE tube. A tapered, pointed, or rounded distal portion is connected to the hollow cylindrical portion. String or extension are attached to the hollow cylindrical portion or the tapered, pointed, or rounded distal portion.

According to yet another embodiment of the Nasal RAE Tube Protective Tip and Method of Use thereof, a method of using a nasal RAE tube having a tube protective tip, includes several steps. The first step is providing a nasal RAE tube with a tube protective sheath or tip. The tube protective sheath or tip has a hollow cylindrical portion extending and fitting snugly over the tube tip. The tube protective sheath or tip further has a tapered, pointed, or rounded distal portion connected to the hollow cylindrical portion. The tube protective sheath or tip further has strings or extensions attached to the hollow cylindrical portion or the tapered, pointed, or rounded distal portion. The second step of the method is introducing the nasal RAE tube with the tube protective sheath or tip into the nasal cavity of a patient. The third step is manipulating the nasal RAE tube with the tube protective sheath or tip until the tube protective sheath or tip is visible in the pharynx at the back of the patient's oral cavity, while retaining the distal ends of the strings or extensions outside of the patient's nasal cavity. The fourth step is inserting a Magill's forceps by way of the patient's mouth. The fifth step is removing the tube protective sheath or tip from the nasal RAE tube using the Magill's forceps and withdrawing it from the patient.

### DESCRIPTION OF THE DRAWINGS

The above-mentioned and other features of embodiments of the RAE tube protective tip and method of use thereof will become more apparent and will be better understood by reference to the following description of embodiments of the RAE tube protective tip and method of use thereof taken in conjunction with the accompanying drawings, wherein:

FIGS. 1A and 18 are side views of a known nasal RAE tubes, as described herein;

FIG. 2 is a side view of a known nasal RAE tube being inserted by way of a patient's nasal passages, as described herein;

FIG. 3 is a side view of a known nasal RAE tube inserted into a patient's trachea, with its cuff inflated, as described herein;

FIG. 4A is a partial side view of a known nasal RAE tube, as described herein;

FIGS. 4B and 4C are partial side views of Nasal RAE Tubes having Protective Tips according to embodiments of the present invention, as described herein;

FIG. 5 is a side view of a Nasal RAE Tube having a Protective Tip according to an embodiment of the present invention, as described herein;

FIG. 6 is a side view of a Nasal RAE Tube having a Protective Tip according to an embodiment of the present invention being inserted by way of a patient's nasal passages, as described herein; and

FIG. 7 is a partial side view of a Nasal RAE Tube having a Protective Tip according to an embodiment of the present invention, as described herein.

Corresponding reference numbers indicate corresponding parts throughout the several views. The exemplifications set out herein illustrate embodiments of the RAE tube protective tip and method of use thereof, and such exemplifications are not to be construed as limiting the scope of the claims in any manner.

### DETAILED DESCRIPTION

The following detailed description and appended drawing describe and illustrate various exemplary embodiments of the invention. The description and drawings serve to enable one skilled in the art to make and use the invention, and are not intended to limit the scope of the invention in any manner. In respect of any methods disclosed and illustrated, the steps presented are exemplary in nature, and thus, the order of the steps is not necessary or critical.

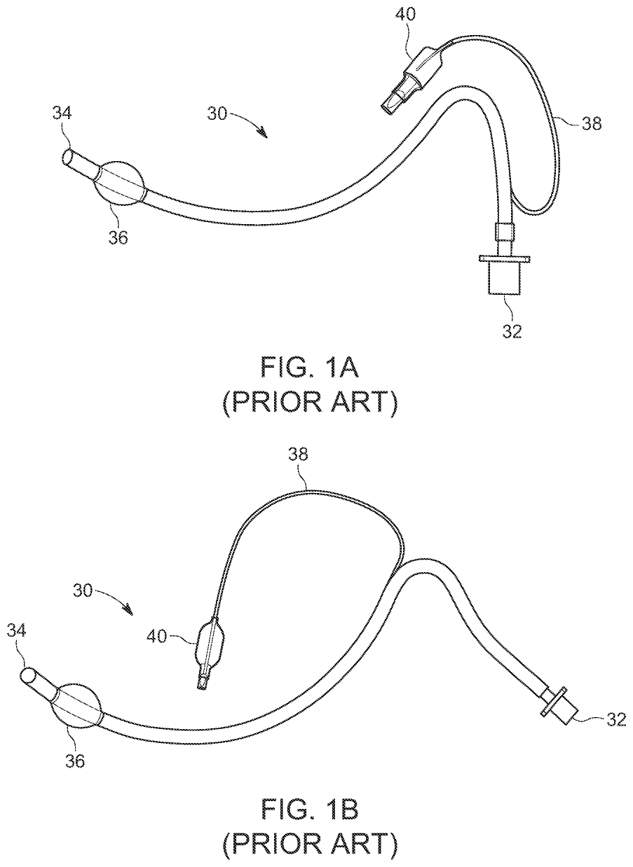

Turning now to FIGS. 1A and 1B, two examples of known nasal Right Angle Endotracheal (RAE) tubes 30 are shown. Each is provided with an air supply connection 32 that is used to connect the nasal RAE tubes 30 to machines that ensure an adequate supply of air to a patient's lungs. At the distal ends, tube tips 34 are proximate to cuffs 36 that are used to seal the nasal RAE tubes 30 within the trachea of the patient. To accomplish this, the cuffs 36 are connected to cuff inflator valves 40 by way of cuff inflator tubes 38 in known fashion. When the cuffs 36 are to be inflated, a syringe (not shown) is connected to the cuff inflator valve 40, so that air is pushed through the cuff inflator valve 40, through the cuff inflator tube 38, and into the cuff 36.

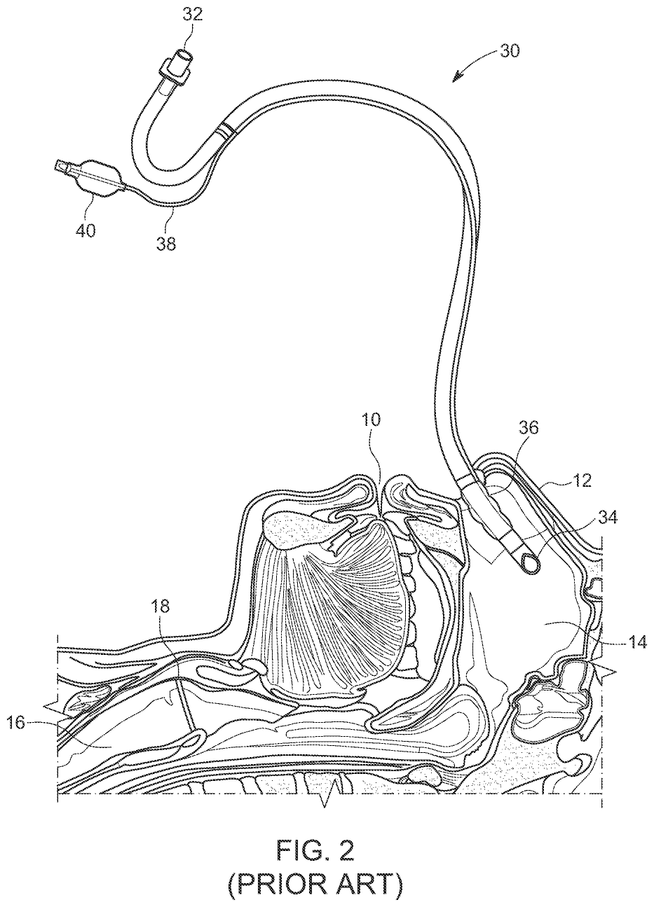

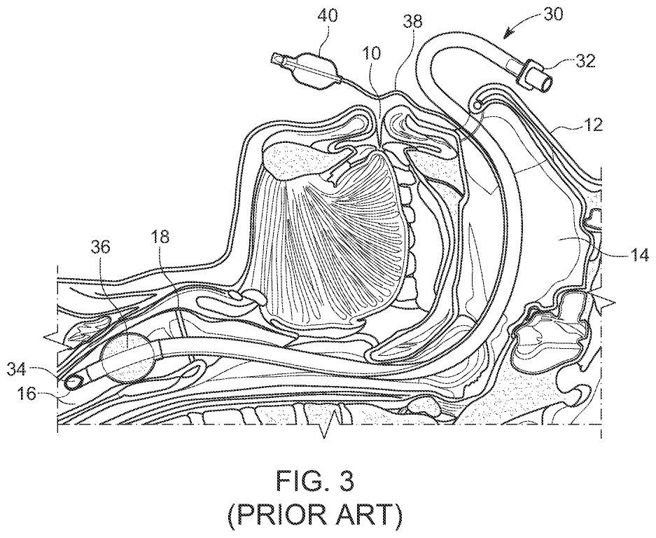

FIG. 2 shows an instance of a known nasal RAE tube 30 being inserted by way of a patient's nose 12 and nasal cavity 14. FIG. 3 shows an instance of a known nasal RAE tube 30 having been inserted by way of the nose 12 and nasal cavity 14, such that the tube tip 34 has passed beyond the vocal cords 18 into the trachea 16. The cuff 36 has been inflated using a syringe (not shown) connected to the the cuff inflator valve 40, which is connected to the cuff 36 by way of the cuff inflator tube 38, so that the tube tip 34 of the nasal RAE tube 30 is secure within the trachea 16. The patient's air is thereby supplied by a machine to which the air supply connection 32 is connected. In the process of inserting the nasal RAE tube 30, it is again necessary for the tube tip 34 of the nasal RAE tube 30 to pass through the patient's nasal cavity 14, as well as through the patient's pharynx, larynx, and/or vocal cords 18, where the excessively rigid, wide, blunt, and/or flared tube tip 34 of the nasal RAE tube 30 may traumatize tissue and cause bleeding. As is known, Magill's forceps (not shown) may sometimes be inserted through the patient's mouth and oral cavity 10 for the purpose of guiding the tube tip 34 of the nasal RAE tube 30 past the vocal cords 18 and into the trachea 16. As noted previously, excessive bleeding caused by the excessively rigid, wide, blunt, and/or flared tube tip 34 of the nasal RAE tube 30 passing through the nasal cavity 14, as well as through the patient's pharynx, larynx, and/or vocal cords 18 may make guiding the tube tip 34 of the nasal RAE tube 30 past the vocal cords 18 and into the trachea 16 difficult or impossible, as well as possibly causing harm to the patient.

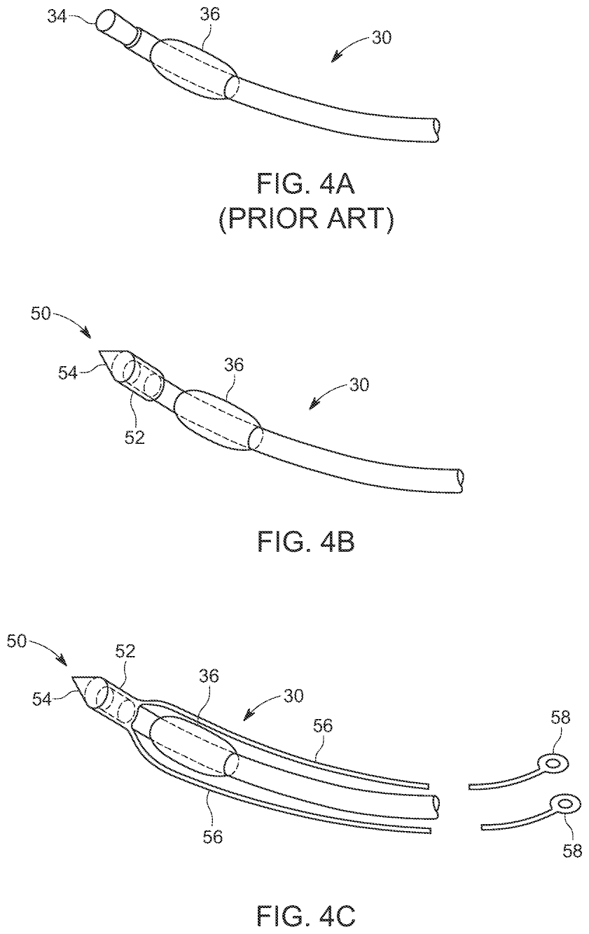

FIG. 4A shows a partial detail view of a known nasal RAE tube 30 having a tube tip 34 and cuff 36. The tube tip 34 of the nasal RAE tube 30 can be seen to be excessively rigid, wide, blunt, and/or flared. FIGS. 48 and 4C, then, each shows an embodiment of the nasal RAE tube protective sheath or tip 50 of the present invention installed on a nasal RAE tube 30 having a tube tip 34 and cuff 36. The nasal RAE tube protective sheath or tip 50 is provided with a hollow cylindrical portion 52 that extends over the tube tip 34 and fits snugly thereto. The nasal RAE tube protective sheath or tip 50 is further provided with a distal portion 54 that is tapered, pointed, or rounded in aspect. As noted previously, the distal portion 54 of the nasal RAE tube protective sheath or tip 50 may be solid or hollow, or a combination of both, as needed to provide the level of stiffness and resiliency required. In this way, as the nasal RAE tube 30 is inserted through the patient's nasal cavity, the nasal RAE tube protective sheath or tip 50 spreads the passages gently and smoothly, allowing the nasal RAE tube 30 to follow it into the pharynx, and minimizing trauma and bleeding of the nasal passages.

As shown in FIG. 4C, strings or extensions 56 may be attached to the nasal RAE tube protective sheath or tip 50 and extend along the nasal RAE tube 30. At their distal ends, the strings or extensions 56 may be provided with loops 58. As noted previously, the strings or extensions 56 and loops 58 are used to ensure that the nasal RAE tube protective sheath or tip 50 does not get lost in the patient's nasal passages if the nasal RAE tube 30 is withdrawn, and/or to ensure that the nasal RAE tube protective sheath or tip 50 does not inadvertently obstruct the trachea during the removal process.

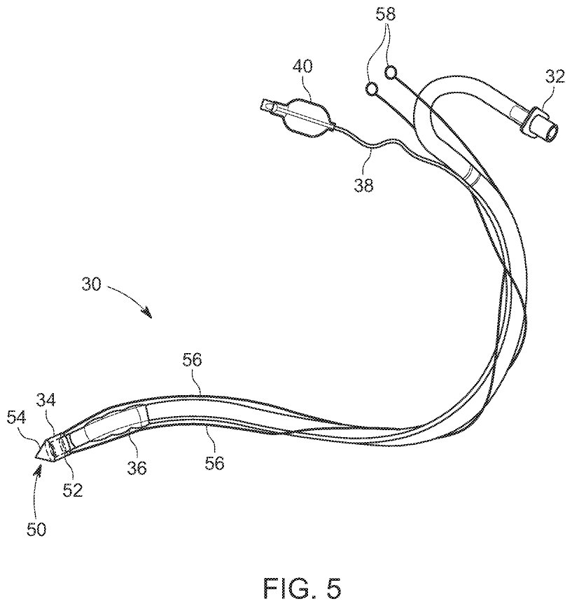

FIG. 5, then, shows a complete nasal RAE tube 30 having an embodiment of the nasal RAE tube protective sheath or tip 50. The nasal RAE tube 30 is again provided with an air supply connection 32 and a tube tip 34 for insertion into the trachea of a patient. A cuff 36 is again connected to a cuff inflator valve 40 by way of a cuff inflator tube 38, and is used to seal the nasal RAE tube 30 against the patient's trachea in the intubated position. The nasal RAE tube protective sheath or tip 50 is again provided with a hollow cylindrical portion 52 that extends over the tube tip 34 and fits snugly thereto. The nasal RAE tube protective sheath or tip 50 is again provided with a distal portion 54 that is tapered, pointed, or rounded in aspect, and strings or extensions 56 having loops 58 connected to the hollow cylindrical portion 52 and/or to the tapered, pointed, or rounded distal portion 54.

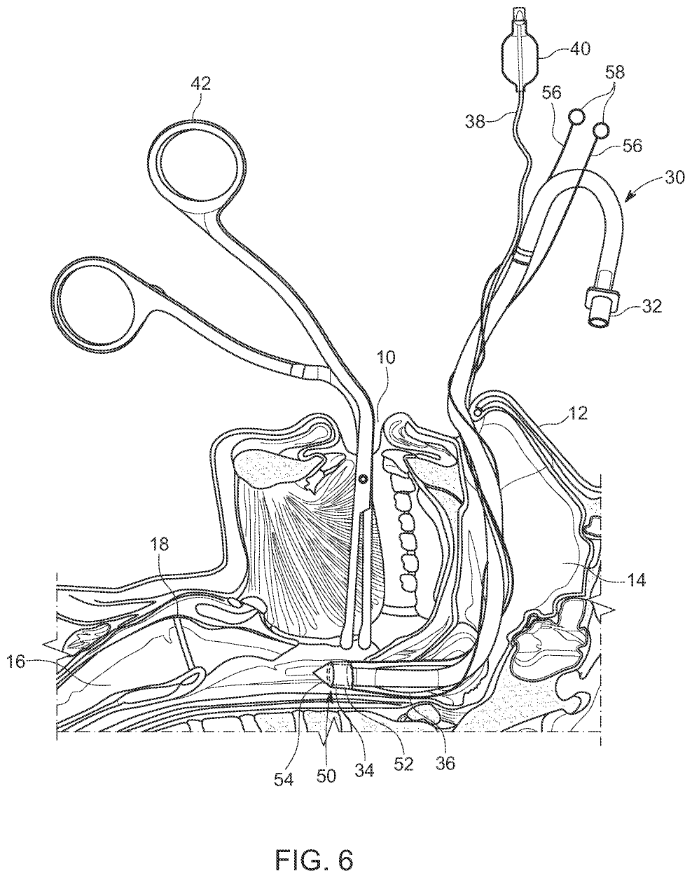

FIG. 6 shows a method of using the nasal RAE tube 30 having an embodiment of the nasal RAE tube protective sheath or tip 50 of the present invention with its hollow cylindrical portion 52 snugly engaged with the tube tip 34 and its tapered, pointed, or rounded distal portion 54. The nasal RAE tube 30 is again provided with an air supply connection 32. The tube tip 34 of the nasal RAE tube 30 with the nasal RAE tube protective sheath or tip 50 in place is introduced into the nasal cavity 14 of the patient by way of the patient's nose 12. The nasal RAE tube 30 with the nasal RAE tube protective sheath or tip 50 in place is then manipulated until the tube tip 34 appears in the pharynx at the back of the patient's oral cavity 10. If it becomes necessary to withdraw the nasal RAE tube 30, as noted previously, then the strings or extensions 56 that extend along the nasal RAE tube 30 and loops 58 are used to ensure that the nasal RAE tube protective sheath or tip 50 is withdrawn along with the nasal RAE tube 30, or minimally subsequent to withdrawal of the nasal RAE tube 30.

As noted previously, once the tube tip 34 appears in the pharynx at the back of the patient's oral cavity 10, the surgeon inserts Magill's forceps 42 by way of the patient's mouth. At this point, the surgeon may decide to remove the nasal RAE tube protective sheath or tip 50 from the tube tip 34. If so, the nasal RAE tube protective sheath or tip 50 may then be withdrawn by way of the patient's oral cavity 10, in which case the strings or extensions 56 and loops 58 follow through the patient's nasal cavity 14. Alternately, the nasal RAE tube protective sheath or tip 50 may then be withdrawn by way of the patient's nasal cavity 14, so that the nasal RAE tube protective sheath or tip 50 travels back through the patient's nasal cavity 14. Still alternately, once the nasal RAE tube protective sheath or tip 50 is secured using the Magill's forceps 42, the surgeon may decide to sever the strings or extensions 56 so that they are removed back through the patient's nasal cavity 14, and the nasal RAE tube protective sheath or tip 50 is removed through the patient's oral cavity 10.

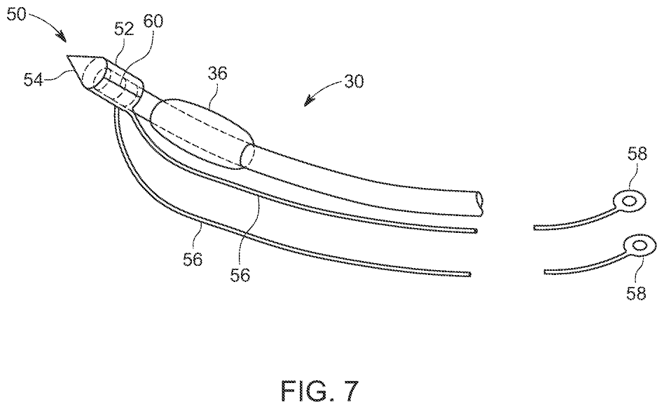

As noted previously, the nasal RAE tube protective sheath or tip 50 may be left in place on the tube tip 34 of the nasal RAE tube 30 until the tube tip 34 passes the vocal cords 18 and enters the trachea 16. Prior to inflation of the cuff 36 using a syringe (not shown), the cuff inflator valve 40, and the cuff inflator tube 38, the nasal RAE tube protective sheath or tip 50 is withdrawn using the strings or extensions 56 and loops 58. In order to facilitate this, as noted previously, an embodiment of the nasal RAE tube protective sheath or tip 50 may, for non-limiting example, be provided with one or more perforations or similar separation features 60, positioned longitudinally along the hollow cylindrical portion 52 as shown in FIG. 7 or otherwise. The strings or extensions 56 in an embodiment of the nasal RAE tube protective sheath or tip 50 may, for non-limiting example, also be attached to of the nasal RAE tube protective sheath or tip 50 offset to one side as shown in FIG. 7. In this way, when the strings or extensions 56 and loops 58 are pulled, using the Magill's forceps 42 or otherwise, the hollow cylindrical portion 52 of the nasal RAE tube protective sheath or tip 50 splits, and the nasal RAE tube protective sheath or tip 50 is withdrawn. In any such embodiment, the nasal RAE tube protective sheath or tip 50 remains intact with its hollow cylindrical portion 52 and its tapered, pointed, or rounded distal portion 54 all connected to the strings or extensions 56, thereby ensuring that the nasal RAE tube protective sheath or tip 50 does not obstruct the trachea 16.

While the RAE tube protective tip and method of use thereof has been described with respect to at least one embodiment, the RAE tube protective tip and method of use thereof can be further modified within the spirit and scope of this disclosure, as demonstrated previously. This application is therefore intended to cover any variations, uses, or adaptations of the RAE tube protective tip and method of use thereof using its general principles. Further, this application is intended to cover such departures from the present disclosure as come within known or customary practice in the art to which the disclosure pertains and which fall within the limits of the appended claims.

### REFERENCE NUMBER LISTING

10 Oral cavity/Mouth
12 Nose
14 Nasal cavity
16 Trachea
18 Vocal cords
30 Nasal Right Angle Endotracheal (RAE) tube
32 Air supply connection
34 Tube tip
36 Cuff
38 Cuff inflator tube
40 Cuff inflator valve
42 Magill's forceps
50 RAE tube protective sheath/tip
52 Hollow cylindrical portion
54 Distal portion
56 Strings/Extensions
58 Loops
60 Perforation or separation feature

What is claimed is:

1. A Tube Protective Sheath or Tip for use with a Nasal Right Angle Endotracheal (RAE) Tube, comprising:
a hollow cylindrical portion configured to extend and fit snugly over a tube tip of a nasal RAE tube;
a tapered, pointed, or rounded distal portion connected to the hollow cylindrical portion; and
at least one string or extension attached to one of the hollow cylindrical portion and the tapered, pointed, or rounded distal portion.

2. The Tube Protective Sheath or Tip of claim 1, wherein:
the hollow cylindrical portion is between 1 and 2 centimeters long; and
the tapered, pointed, or rounded distal portion is between 1 and 3 centimeters long.

3. The Tube Protective Sheath or Tip of claim 1, wherein:
the Tube Protective Sheath or Tip being provided in different colors coded to indicate the size or diameter of nasal RAE tube with which the Tube Protective Sheath or Tip is configured to engage; and
each of the different colors chosen for color coding being chosen to provide color contrast with blood.

4. The Tube Protective Sheath or Tip of claim 1, wherein:
the tapered, pointed, or rounded distal portion being solid, hollow, or a combination of solid and hollow.

5. The Tube Protective Sheath or Tip of claim 4, wherein:
a hollow space within the tapered, pointed, or rounded distal portion being in fluid communication with the interior of the hollow cylindrical portion.

6. The Tube Protective Sheath or Tip of claim 1, wherein:
the hollow cylindrical portion being further configured to extend partially or fully over a cuff of the nasal RAE tube.

7. The Tube Protective Sheath or Tip of claim 1, wherein:
the Tube Protective Sheath or Tip being made from silicone, rubber, or plastic.

8. The Tube Protective Sheath or Tip of claim 1, wherein:
the hollow cylindrical portion having one or more separation features; and
the at least one string or extension being attached to the hollow cylindrical portion or the tapered, pointed, or rounded distal portion asymmetrically.

9. The Tube Protective Sheath or Tip of claim 1, wherein:
the Tube Protective Sheath or Tip being provided pre-lubricated in a package.

10. A Nasal RAE Tube having a Tube Protective Sheath or Tip, comprising:
a tube;
an air supply connection connected to the tube;
a tube tip for insertion into the trachea of a patient by way of the patient's nasal cavity;
a cuff for inflatably sealing against the patient's trachea;
a cuff inflator valve connected to the cuff by way of a cuff inflator tube; and
a tube protective sheath or tip having:
a hollow cylindrical portion configured to extend and fit snugly over the tube tip;
a tapered, pointed, or rounded distal portion connected to the hollow cylindrical portion; and
at least one string or extension attached to one of the hollow cylindrical portion and the tapered, pointed, or rounded distal portion.

11. The Nasal RAE Tube of claim 10, wherein:
the hollow cylindrical portion is between 1 and 2 centimeters long; and
the tapered, pointed, or rounded distal portion is between 1 and 3 centimeters long.

12. The Nasal RAE Tube of claim 10, wherein:
the tube protective sheath or tip being provided in a color being chosen to provide color contrast with blood.

13. The Nasal RAE Tube of claim 10, wherein:
the tapered, pointed, or rounded distal portion being solid, hollow, or a combination of solid and hollow.

14. The Nasal RAE Tube of claim 13, wherein:
a hollow space within the tapered, pointed, or rounded distal portion being in fluid communication with the interior of the hollow cylindrical portion.

15. The Nasal RAE Tube of claim 10, wherein:
the hollow cylindrical portion being further configured to extend partially or fully over the cuff.

16. The Nasal RAE Tube of claim 10, wherein:
the tube protective sheath or tip being made from silicone, rubber, or plastic.

17. The Nasal RAE Tube of claim 10, wherein:
the hollow cylindrical portion having one or more separation features; and
the at least one string or extension being attached to the hollow cylindrical portion or the tapered, pointed, or rounded distal portion asymmetrically.

18. A method of using a Nasal RAE Tube having a Tube Protective Tip, comprising the steps of:
providing a nasal RAE tube with a tube protective sheath or tip, the tube protective sheath or tip having:
a hollow cylindrical portion extending and fitting snugly over the tube tip;
a tapered, pointed, or rounded distal portion connected to the hollow cylindrical portion; and
at least one string or extension attached to one of the hollow cylindrical portion and the tapered, pointed, or rounded distal portion.
introducing the nasal RAE tube with the tube protective sheath or tip into the nasal cavity of a patient;
manipulating the nasal RAE tube with the tube protective sheath or tip until the tube protective sheath or tip is visible in the pharynx at the back of the patient's oral cavity, while a distal end of the at least one string or extension remains outside of the patient's nasal cavity;
inserting a Magill's forceps by way of the patient's mouth; and
removing the tube protective sheath or tip from the nasal RAE tube using the Magill's forceps and withdrawing it from the patient.

19. The method of claim 18, further comprising the step(s) of:
severing the at least one string or extension from the hollow cylindrical portion or the tapered, pointed, or rounded distal portion prior to withdrawing the tube protective sheath or tip from the patient.

19. The method of claim 18, further comprising the step(s) of:
providing the tube protective sheath or tip with one or more separation features;
attaching the at least one string or extension to the hollow cylindrical portion or the tapered, pointed, or rounded distal portion asymmetrically;
manipulating the nasal RAE tube with the tube protective sheath or tip until the tube protective sheath or tip advances past the patient's vocal cords into the patient's trachea;
pulling on the at least one string or extension using the Magill's forceps until the one or more separation features separate; and
withdrawing the tube protective sheath or tip from the patient.
